(19) United States
(12) Patent Application Publication
(10) Pub. No.: US 2008/0139838 A1
(43) Pub. Date: Jun. 12, 2008

(54) Method for Producing Bi-Functional Ammonium Nitriles

(76) Inventors: Gerd Reinhardt, Kelkheim (DE); Hans Prehler, Kriftel (DE); Lars Cuypers, Munchen (DE)

Correspondence Address: CLARIANT CORPORATION;INTELLECTUAL PROPERTY DEPARTMENT, 4000 MONROE ROAD, CHARLOTTE, NC, 28205, US

(21) Appl. No.: 11/885,609

(22) PCT Filed: Feb. 24, 2006
(86) PCT No.: PCT/EP06/01733

§ 371 Date: Jan. 4, 2008

(30) Foreign Application Priority Data
Mar. 1, 2005 (DE) 10 2005 009 136.9

(51) Int. Cl. C07C 255/24

(52) U.S. Cl. 558/467

(57) ABSTRACT

The invention relates to a method for producing bifunctional ammonium nitriles of the following general formulae:

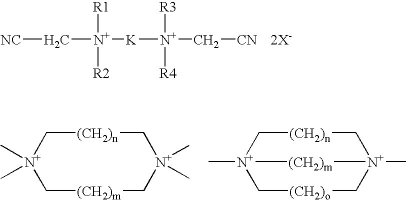

by reacting a tertiary diamine with a cyanomethylene-group-transferring quaternizing agent in a polar, aprotic solvent. Bifunctional ammonium nitriles may be used as bleach activators in detergents and cleaners.

The present invention relates to an improved synthesis of bifunctional ammonium nitrites through single-stage reaction of tertiary diamines with quaternizing agents.

The patent literature describes ammonium nitriles and their use as bleach activators in detergents and cleaners. Examples which may be listed are EP-A-303 520, EP-A458 396, EP-A-464 880 and WO-A-2 003 078 561. By adding the activators, the bleaching effect of aqueous peroxide solutions can be increased to such a degree that, at 40° C., essentially the same effect arises as is otherwise achieved only with the peroxide solution on its own at 95° C.

EP-A-303 520 describes, inter alia, the synthesis of the bifunctional ammonium nitrites (I) and (II):

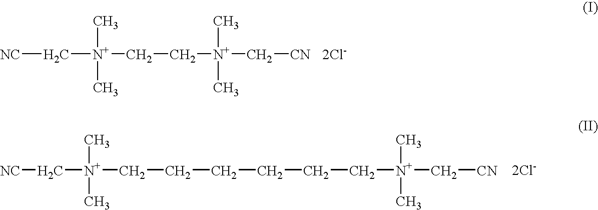

According to EP-A-303 520, these ammonium nitriles are obtained by reacting chloroacetonitrile with an excess of N,N,N′,N′-tetramethylethylene-diamine or N,N,N′,N′-tetramethylhexane-1,6-diamine in acetone at 90° C. When reworking the described syntheses for (I) and (II), only unsatisfactory yields could be obtained. For example, compound (I) was isolated only with a yield of 11%.

It was therefore the object to find an improved method for producing bifunctional ammonium nitriles.

Surprisingly, it has been found that, by reacting tertiary diamines with cyanomethylene-group-transferring quaternizing agents, bifunctional ammonium nitriles are obtained in very pure form and in high yields.

The present invention thus provides a method for the synthesis of compounds of the general formula

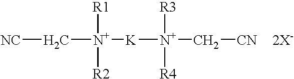

where K is a group of the formulae

—(CH₂)ₙ—, —(CH₂)ₙ-A-(CH₂)ₙ—,

A is oxygen or a group of the formula N—R⁵,
R¹, R², R³ and R⁴, in each case independently of one another, are a straight-chain or branched-chain C₁- to C₂₄-alkyl, alkenyl or alkyl ether group, preferably a C₁- to C₁₈-alkyl, alkenyl or alkyl ether group, or
R¹, R³ and K, together with the two N atoms, are either a group of the formula

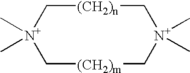

or R¹, R², R³, R⁴ and K, together with the two N atoms, are a group of the formula

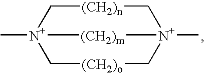

R⁵ is hydrogen or a C₁- to C₂₄-alkyl, alkenyl or cycloalkyl group, preferably a C₁- to C₁₈-alkyl, alkenyl or cycloalkyl group,
X⁻ is an anion, for example chloride, bromide, iodide, toluenesulfonate, benzenesulfonate, cumenesulfonate or mesitylsulfonate and
the variables m, n and o are integers from 1 to 16.

This method consists in reacting a tertiary diamine of the formula

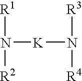

with a cyanomethylene-group-transferring quaternizing agent of the general formula

X—CH₂CN

in a polar, aprotic solvent with a boiling point above 60° C., where A, R¹, R², R³, R⁴, R⁵, X, m, n and o have the meanings given above.

For the method according to the invention, the procedure specifically involves firstly dissolving or suspending the cyanomethylene-group-transferring quaternizing agent in a suitable polar-aprotic solvent. The solvent should have a boiling point above 60° C. Suitable solvents are, for example: ethyl acetate, n-propyl acetate, isopropyl acetate, n-butyl acetate, isobutyl acetate and mixtures thereof, dimethyl sulfoxide, N-methyl-pyrrolidone, 1,3-dimethylimidazolidin-2-one. Preference is given to ethyl acetate, dimethylformamide and dimethylacetamide, and particular preference is given to dimethylacetamide since the starting materials have very good solubility in this solvent, whereas the end products are sparingly soluble and precipitate out. A tertiary diamine (III) is added dropwise to this solution or suspension. The amount of quaternizing agent is 1.8 to 3.0, preferably 2.0 to 2.5, in particular 2.0 to 2.3, mol equivalents, based on the tertiary diamine (III). Suitable quaternizing agents are haloacetonitriles and arylacetonitriles, preference being given to chloroacetonitrile, bromoacetonitrile, iodoacetonitrile, tosylacetonitrile and cumeneacetonitrile, and particular preference being given to chloroacetonitrile. The reaction temperature is generally 20 to 120° C., preferably 30 to 100° C., particularly preferably 40 to 80° C. The reaction runs in a period of from 1 to 10 hours, preferably 2 to 9 hours, particularly preferably 3 to 8 hours. The resulting product can be separated off from the solvent by filtration, suction filtration, decantation or by centrifugation.

The examples below are intended to illustrate the invention in more detail, without limiting it thereto.

### EXAMPLE 1

### Synthesis of N,N,N′,N′-tetramethyl-N,N′-di(cyanomethyl)-1,2-ethanediammonium dichloride

37.75 g (0.5 mol) of chloroacetonitrile were initially introduced into 100 ml of ethyl acetate, and 29 g (0.25 mol) of N,N,N′,N′-tetramethylenediamine were added dropwise with stirring at room temperature. The reaction mixture was stirred for 5 hours at 50° C. and then cooled to room temperature. The precipitated solid was filtered off and washed with three times 50 ml of ethyl acetate. The filter cake was dried under reduced pressure at 60° C. This gave 60.9 g (0.23 mol) of N,N,N′,N′-tetramethyl-N,N′-di(cyanomethyl)-1,2-ethanediammonium dichloride as white solid, corresponding to a yield of 91%.

m.p.: 183° C. (decomposition)

Elemental Analysis:

Found: C, 44.8%; H, 7.5%; N, 21.2%; Cl, 26.5%.

Calculated: C, 45.0%; H, 7.5%; N, 21.0%; Cl, 26.5%.

¹H NMR (D₂O):

δ=4.75 (4H, s); δ=4.34 (4H, s); δ=3.54 (12H, s)

IR (KBr):

3040 vs, 3020 vs, 2960 vs, 1475 vs, 1440 s, 1405 m, 1350 w, 1300 w, 1250 vw, 1215 vw, 1130 w, 1010 w, 985 vs, 920 vs, 910 s, 795 s, 755 vw

### EXAMPLE 2

### Synthesis of N,N,N′,N′-tetramethyl-N,N′-di(cyanomethyl)-1,3-propanediammonium dichloride

57.38 g (0.76 mol) of chloroacetonitrile were initially introduced into 160 ml of N,N-dimethylacetamide, and 50 g (0.38 mol) of N,N,N′,N′-tetramethyl-1,3-propanediamine were added dropwise with stirring at room temperature. The reaction mixture was stirred for 5 hours at 50° C. and then cooled to room temperature. The precipitated solid was filtered off and washed with N,N-dimethylacetamide. The filter cake was dried at 60° C. under reduced pressure. This gave 100.3 g (0.36 mol) of N,N,N′,N′-tetramethyl-N,N′-di(cyanomethyl)-1,3-propanediammonium dichloride as white solid, corresponding to a yield of 94%.

m.p.: 153° C.

¹H NMR (D₂O):

δ=4.75 (4H, s); δ=3.74 (4H, t); δ=3.44 (12H, s); δ=2.54 (2H, m)

### EXAMPLE 3

### Synthesis of N,N,N′,N′-tetraethyl-N,N′-di(cyanomethyl)-1,3-propanediammonium dichloride

19.6 g (0.26 mol) of chloroacetonitrile were initially introduced into 75 ml of N,N-dimethylacetamide, and 25 g (0.13 mol) of tetraethyl-1,3-propanediamine were added dropwise with stirring at room temperature. The reaction mixture was stirred for 7 hours at 55° C. and then cooled to room temperature. The precipitated solid was filtered off and washed with N,N-dimethylacetamide. The filter cake was dried at 60° C. under reduced pressure. This gave 18.3 g (0.05 mol) of N,N,N′,N′-tetraethyl-N,N′-di(cyanomethyl)-1,3-propanediammonium dichloride as white solid, corresponding to a yield of 42%.

m.p.: 203° C. (decomposition)

¹H NMR (D₂O):

δ=4.75 (4H, s); δ=3.72-3.65 (12H, m); δ=2.38 (2H, m); δ=1.44 (12H, t)

### EXAMPLE 4

### Bleaching Power of Bifunctional Ammonium Nitriles

The bleaching power of the cyanomethylammonium salts was investigated in a Linitest instrument (Heraus) at 20, 40 and 60° C. For this, 2 g/l of a bleach-free basic detergent (WMP, WFK, Krefeld) and 0.5 g/l of sodium perborate monohydrate (Degussa) were dissolved in water of hardness level 3. Then, either 100 mg/l, 200 mg/l or 250 mg/l of activator were added. The washing time was 30 min. The sections of fabric were then rinsed with water, dried and ironed. Tea BC-1 and Curry BC-4 (WFK Testgewebe GmbH, Krefeld) on cotton served as bleaching test fabric. The bleaching result evaluated was the difference in reflectance, measured using an Elrepho instrument, after washing compared to the fabric washed with 2 g/l of WMP and 0.5 g/l of sodium perborate monohydrate.

ΔΔR=ΔR(formulation+persalt+activator)−ΔR(formulation*persalt)

Bleach compositions were prepared and tested with the cationic nitrile compounds 1, 2 and 3 according to the invention, and the comparison substances 4.

The compounds 1 to 4 are

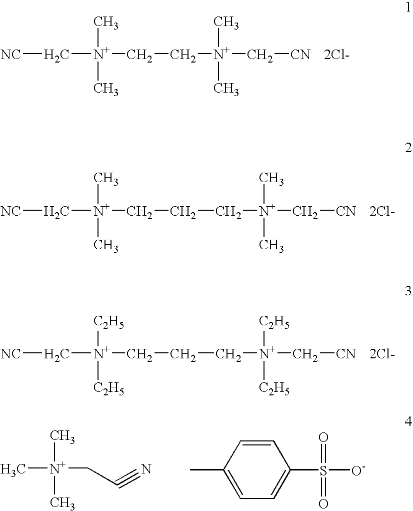

With the activators 1 and 3, washing experiments were carried out on the bleaching test fabrics Tea BC-1 and Curry BC-4 at 20, 40 and 60° C. at concentrations of from 0.1 and 0.2 g/l. The results are shown in table 1:

TABLE 1
Test results (ΔΔR values) for the activators 1 and 3 on Tea BC-1 and Curry BC-4

| | Activator 1 | Activator 3 | Activator 1 | Activator 3 |
|---|---|---|---|---|
| Washing conditions | Tea BC-1 | | Curry BC-4 | |
| 20° C.; c (activator) = 0.1 g/l | 9.6 | 6.9 | 3.8 | 2.9 |
| 20° C.; c (activator) = 0.2 g/l | 14.2 | 9.4 | 4.0 | 3.9 |
| 40° C.; c (activator) = 0.1 g/l | 8.5 | 10.2 | 2.9 | 4.3 |
| 40° C.; c (activator) = 0.2 g/l | 13.9 | 10.3 | 3.2 | 4.1 |
| 60° C.; c (activator) = 0.1 g/l | 5.3 | 2.8 | 2.2 | 1.2 |
| 60° C.; c (activator) = 0.2 g/l | 7.1 | 5.7 | 1.8 | −0.4 |

For the activators 1 and 2, washing experiments were carried out at 20 and 40° C. at concentrations of 0.1 or 0.2 g/l. The results are shown in table 2:

TABLE 2
Test results (ΔΔR values) for the activators 1 and 2 on Tea BC-1 and Curry BC-4

| | Activator 1 | Activator 2 | Activator 1 | Activator 2 |
|---|---|---|---|---|
| Washing conditions | Tea BC-1 | | Curry BC-4 | |
| 20° C.; c (activator) = 0.1 g/l | 10.6 | 9.8 | 4.1 | 4.7 |
| 20° C.; c (activator) = 0.2 g/l | 14.9 | 14.5 | 5.4 | 5.7 |
| 40° C.; c (activator) = 0.1 g/l | 11.5 | 12.5 | 3.4 | 4.1 |
| 40° C.; c (activator) = 0.2 g/l | 14.3 | 16.5 | 3.5 | 4.4 |

Furthermore, with activator 1, washing experiments were carried out at 20 or 40° C. at a concentration of 0.25 g/l. The results are compared in table 3 with the hydrophobic activator 4:

TABLE 3
Test results (ΔΔR values) for activators 1 and
4 at 20 and 40° C. on various test soilings

| | Activator 1 | Activator 4 | Activator 1 | Activator 4 |
|---|---|---|---|---|
| | 20° C. | | 40° C. | |
| Blackcurrant | 1.7 | 1.8 | 0.9 | 0.6 |
| Chlorophyll | 3.0 | 0.1 | 2.0 | 1.1 |
| Red wine CS-3 | 13.6 | 4.6 | 10.8 | 5.6 |
| Grass | 9.4 | 2.3 | 8.4 | 2.2 |
| Ketchup | 3.0 | 0.6 | 1.3 | 1.0 |
| Tea BC-3 | 25.3 | 8.4 | 23.6 | 12.5 |

1. A method for producing bifunctional ammonium nitrites of the formula 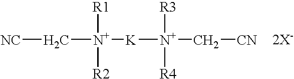
where K is a group of the formulae
—(CH₂)ₙ—, —(CH₂)ₙ-A-(CH₂)ₙ—,
A is oxygen or a group of the formula N—R⁵,
R¹, R², R³ and R⁴, in each case independently of one another, are a straight-chain or branched-chain C₁- to C₂₄-alkyl, alkenyl or alkyl ether group, preferably a C₁- to C₁₈-alkyl, alkenyl or alkyl ether group, or
R¹, R³ and K, together with the two N atoms, are either a group of the formula 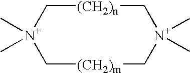
or R¹, R², R³, R⁴ and K, together with the two N atoms, are a group of the formula 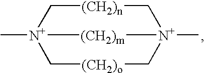
R⁵ is hydrogen or a C₁- to C₂₄-alkyl, alkenyl or cycloalkyl group, preferably a C₁- to C₁₈-alkyl, alkenyl or cycloalkyl group,
X⁻ is an anion, for example chloride, bromide, iodide, toluenesulfonate, benzenesulfonate, cumenesulfonate or mesitylsulfonate and
the variables m, n and o are integers from 1 to 16,
said method comprising reacting a tertiary diamine of the formula 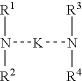
with a cyanomethylene-group-transferring quaternizing agent of the general formula
X—CH₂CN
in a polar, aprotic solvent with a boiling point above 60° C., where A, R¹, R², R³, R⁴, R⁵, X, m, n and o have the meanings given above.

2. The method as claimed in claim 1, wherein said quaternizing agent of the formula X—CH₂CN is a haloacetonitrile or an arylacetonitrile or a mixture thereof.

3. The method as claimed in claim 1, wherein said quaternizing agent is selected from the group consisting of chloroacetonitrile, bromoacetonitrile, iodoacetonitrile, tosylacetonitrile and cumeneacetonitrile.

4. The method as claimed in claim 1, wherein said quaternizing agent is chloroacetonitrile.

5. The method as claimed in claim 1, wherein said quaternizing agent is present in an amount of 1.8 to 3.0, based on the tertiary diamine.

6. The method as claimed in claim 1, wherein said quaternizing agent is present in an amount of 2.0 to 2.5, based on the tertiary diamine.

7. The method as claimed in claim 1, wherein said quaternizing agent is present in an amount of 2.0 to 2.3, based on the tertiary diamine.

8. The method as claimed in claim 1, wherein said polar, aprotic solvent is selected from the group consisting of ethyl acetate, n-propyl acetate, isopropyl acetate, n-butyl acetate, isobutyl acetate, dimethyl sulfoxide, N-methylpyrrolidone, 1,3-dimethylimidazolidin-2-one, and mixtures thereof.

9. The method as claimed in claim 1, wherein said reacting step is carried out in the presence of said solvent selected from the group consisting of ethyl acetate, dimethylformamide and dimethylacetamide.

10. The method as claimed in claim 1, wherein said solvent is dimethylacetamide.

11. The method as claimed in claim 1, wherein said reacting is carried out at a temperature of 20 to 120° C.

12. The method as claimed in claim 1, wherein said reacting is carried out at a temperature of 30 to 100° C.

13. The method as claimed in claim 1, wherein said reacting is carried out at a temperature of 40 to 80° C.

14. The method as claimed in claim 1, wherein the reacting includes a reaction time of 1 to 10 hours.

15. The method as claimed in claim 1, wherein the reacting includes a reaction time of 2 to 9 hours.

16. The method as claimed in claim 1, wherein the reacting includes a reaction time of 3 to 8 hours.

17. A detergent, cleaner or disinfectant comprising a bifunctional ammonium nitrile produced as claimed in claim 1.

18. A machine dishwashing composition comprising a bifunctional ammonium nitrile produced as claimed in claim 1.

19. A detergent formulation comprising a bifunctional ammonium nitrile produced as claimed in claim 1.
